(19) United States
(12) Patent Application Publication
(10) Pub. No.: US 2009/0082788 A1
(43) Pub. Date: Mar. 26, 2009

(54) SUTURE MANAGEMENT METHOD AND APPARATUS

(76) Inventor: Amr ElMaraghy, Toronto (CA)

Correspondence Address: BERESKIN AND PARR, 40 KING STREET WEST, BOX 401, TORONTO, ON, M5H 3Y2, CA

(21) Appl. No.: 12/199,530

(22) Filed: Aug. 27, 2008

Related U.S. Application Data
(60) Provisional application No. 60/974,911, filed on Sep. 25, 2007.

(51) Int. Cl. A61B 17/04

(52) U.S. Cl. 606/148

(57) ABSTRACT

An instrument for minimally invasive surgery includes a shank attachable to a handle, the shank extending lengthwise along a shank axis between a handle end and a tip end; and a tip joined to the tip end of the shank, the tip comprising a slender, elongate body extending between a proximal end adjacent the tip and of the shank and a distal end opposite the proximal end. The tip includes a jaw member adjacent the distal end, the jaw member movable between open and closed positions. The tip includes an outer surface that converges, at least when the jaw member is in the closed position, to a sharp point opposite the tip end of the shank to facilitate penetration through tissue. The tip has a tip length extending between the proximal end and the point, the tip including a compound curve segment along at least a portion of the tip length. The instrument further includes an actuator coupled to the jaw member and extending lengthwise of the instrument to the handle end of the shank.

This application claims the benefit of Provisional Application No. 60/974,911, filed Sep. 25, 2007, which is hereby incorporated herein by reference.

### FIELD

The teaching disclosed herein relates to minimally invasive surgery techniques and instruments.

### BACKGROUND

U.S. Pat. No. 5,254,130 (Poncet et al.) discloses a surgical device comprising (a) a tubular housing having a longitudinal bore extending therethrough, (b) a first elongate member extending through the longitudinal bore of the housing, and having a proximal and a distal segment, where at least part of the distal segment comprises an elastic material, especially a super elastic material, and wherein the distal segment assumes a first shape when extended from the longitudinal bore, and a second shape when withdrawn into the bore, (c) a second elongate member also having a proximal and a distal segment, the second elongate member extending substantially parallel to the first elongate member so that it is moved by the first elongate member, when the first elongate member changes from its first to its second shape and vice versa, the second elongate member being rotatable relative to, and substantially about, the axis of the first elongate member, and (d) an operating head secured to the distal segment of the second elongate member so that it (i) is moved with the second elongate member when the first elongate member changes from its first to its second shape and (ii) can be rotated by the second elongate member substantially about the axis of the first elongate member.

U.S. Pat. No. 5,899,911 (Carter) discloses a surgical instrument, guide, and method capable of being used for closure of peritoneum fascia, occlusion of bleeding vessels such as inferior epigastric, and for all uses related to accurately passing suture material through a guide into tissue. A tip of a surgical instrument in a standard suture-/needle-driving position with a sharp tip that opens and closes with the surgeon grasping suture material with the sharp tip is provided. Insertion of the tip/suture through tissue until the tip is seen through the peritoneum by direct vision begins the wound-closing procedure. The suture is released by opening and withdrawing the tip from the guide. The suture is recovered by using the guide to redirect the tip and puncturing the tissue opposite the first point of insertion. The tip grasps the suture and pulls the suture through the guide. The suture is pulled outside the wound, providing for rapid closure of the surgical incision. The guide is insertable within the wound to be closed and guides the surgical instrument at a predetermined angle from the longitudinal axis of the guide for optimum wound closure. The surgical instrument and method may be used to advantageously shorten and strengthen ligaments. For example, by gathering and reinforcing the round ligament that supports a woman's uterus with suture materials, surgeons can reposition and stabilize a retroverted uterus.

U.S. Pat. No. 6,074,403 (Nord) discloses an instrument for retrieving suture within a patient, the instrument having a shaft with proximal and distal ends. The distal end terminates in a sharp tip. An opening in the shaft is provided proximal to the sharp tip. A jaw disposed on the shaft captures suture within the opening in the shaft. A hand mechanism disposed on the proximal end of the shaft opens and closes the jaw.

U.S. Pat. No. 6,159,200 (Verdura et al.) discloses systems, methods, and miniature instruments for minimally invasive surgical procedures. A miniature surgical instrument can be inserted directly into a body cavity through a cannula in a way that a surgeon can insert his or her hand into the cavity through a separate minimal incision and use the miniature instruments. A miniature surgical system can include: a cannula having a hollow body configured to receive a miniature surgical instrument and a tether connected to the instrument; and a plunger sized to engage a proximal end of the body and including a conduit therethrough for receiving the tether. The system can also include the tether and the instrument. During use, the instruments can be quickly and safely removed from the body cavity to enable the surgeon to use his or her fingers, e.g., to manipulate tissue.

### SUMMARY

The following summary is intended to introduce the reader to this specification but not to define any invention. In general, this specification discusses one or more methods or apparatuses related to suture management in minimally invasive surgical procedures.

According to one aspect, an instrument for minimally invasive surgery includes a shank attachable to a handle, the shank extending lengthwise along a shank axis between a handle end and a tip end; and a tip joined to the tip end of the shank, the tip comprising a slender, elongate body extending between a proximal end adjacent the tip end of the shank and a distal end opposite the proximal end. The tip includes a jaw member adjacent the distal end, the jaw member movable between open and closed positions. The tip includes an outer surface that converges, at least when the jaw member is in the closed position, to a sharp point opposite the tip end of the shank to facilitate penetration through tissue. The tip has a tip length extending between the proximal end and the point, the tip including a compound curve segment along at least a portion of the tip length. The instrument further includes an actuator coupled to the jaw member and extending lengthwise of the instrument to the handle end of the shank.

According to another aspect, an instrument for minimally invasive surgery includes a shank attachable to a handle, the shank extending lengthwise along a shank axis between a handle end and a tip end; a tip member joined to the tip end of the shank, the tip member extending along a tip length between a proximal end adjacent the tip end of the shank and a distal end opposite the proximal end, the tip member comprising a jaw adjacent the distal end, the jaw movable between open and closed positions, the tip member comprising an outer surface that converges, at least when the jaw member is in the closed position, to a sharp point at the distal end to facilitate penetration through tissue, the tip member being formed in the shape of a compound curve along at least a portion of the tip length; and an actuator coupled to the jaw member and extending lengthwise of the instrument to the handle end of the shank.

In some examples, the compound curve comprises at least a portion of a helix defining a helical portion of the tip. The helical portion can have a helix axis generally collinear with the shank axis. The helical portion can have a helix angle in the range from about 15 degrees to about 90 degrees, and can in some examples be about 45 degrees. The helical portion can have a helix radius about 10 mm or less or within the range of about 4 mm to about 10 mm. The helical portion can extend circumferentially about 180 degrees about the shank axis. The helical portion can comprise the jaw member. The helical portion can comprise the pointed distal end of the tip member. The jaw member can have an outer jaw surface that is tapered at one end, the pointed distal end of the tip member comprising said tapered one end. The tip member can comprise a generally tubular body extending between the proximal end and the jaw member. The actuator can be housed within the tubular body. The actuator can be movable with respect to the tip for moving the jaws between the open and closed positions. The actuator can be slidable relative to the tip. The actuator can comprise an elongate element generally inextensible along its length and laterally flexible.

According to another aspect, an instrument for minimally invasive surgery includes a shank extending lengthwise along a longitudinal axis between a handle end and a tip end; a tip joined to the tip end of the shank, the tip comprising a slender body extending between a proximal end adjacent the tip end of the shank and a distal end opposite the proximal end, at least a portion of the body curved along a helical path defining a helical portion of the tip, the tip comprising a jaw member adjacent the distal end, the jaw member movable between open and closed positions, the tip comprising an outer surface that converges, at least when the jaw member is in the closed position, to a sharp point opposite the tip end of the shank to facilitate penetration through tissue; and an actuator coupled to the jaw member and extending lengthwise of the instrument to the handle end of the shank.

In some examples, the helical portion of the tip can comprise the jaw member. The instrument can include a handle attachable to the handle end of the shank. The handle can include a driver element mounted to the handle and coupled to the actuator, the driver element movable between advanced and retracted positions corresponding to the closed and open positions of the jaws. The driver element can comprise a lever pivotably secured to the handle. The lever can be generally flush against handle body when the jaws are in the closed position.

Other aspects and features of the Applicant's teaching will become apparent, to those ordinarily skilled in the art, upon review of the following description of specific examples.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of a suturing instrument;

FIG. 2 is a cross-sectional view of the instrument of FIG. 1 taken along the lines 2-2;

FIG. 3 is a top view of a tip portion of the instrument of FIG. 1;

FIG. 4 is an end view of the tip portion of FIG. 3;

FIGS. 5 and 6 are bottom, and side views, respectively, of the tip portion of FIG. 3;

FIG. 7 is a cross-sectional view of the top portion of FIG. 5, taken along the lines 7-7;

FIG. 8 is a side view of a jaw element of the instrument of FIG. 1;

FIG. 9 is a partially exploded perspective view of the instrument of FIG. 1;

FIG. 10 is a cross-sectional view of the instrument of FIG. 9 taken along the lines 10-10;

FIG. 11 is a cross-sectional view showing the instrument 100 in use for repairing tissue;

FIG. 12 is a perspective view of another example of a suturing instrument;

FIG. 13 is an enlarged perspective view of a handle portion of the instrument of FIG. 12;

FIG. 14 is a perspective view of another example of a suturing instrument;

FIG. 15 is an enlarged view of a tip portion of the instrument of FIG. 14;

FIGS. 16, 17, and 18 are top, side, and bottom views of a tip portion of the instrument of FIG. 14;

FIG. 19 is an end view of a tip portion of the instrument of FIG. 14; and

FIG. 20 is a perspective view of a tip portion of another example of a suturing instrument.

### DETAILED DESCRIPTION

Various apparatuses or processes will be described below to provide an example of an embodiment of each claimed invention. No embodiment described below limits any claimed invention and any claimed invention may cover processes or apparatuses that are not described below. The claimed inventions are not limited to apparatuses or processes having all of the features of any one apparatus or process described below or to features common to multiple or all of the apparatuses described below. It is possible that an apparatus or process described below is not an embodiment of any claimed invention. The applicants, inventors or owners reserve all rights that they may have in any invention disclosed in an apparatus or process described below that is not claimed in this document, for example the right to claim such an invention in a continuing application and do not intend to abandon, disclaim or dedicate to the public any such invention by its disclosure in this document.

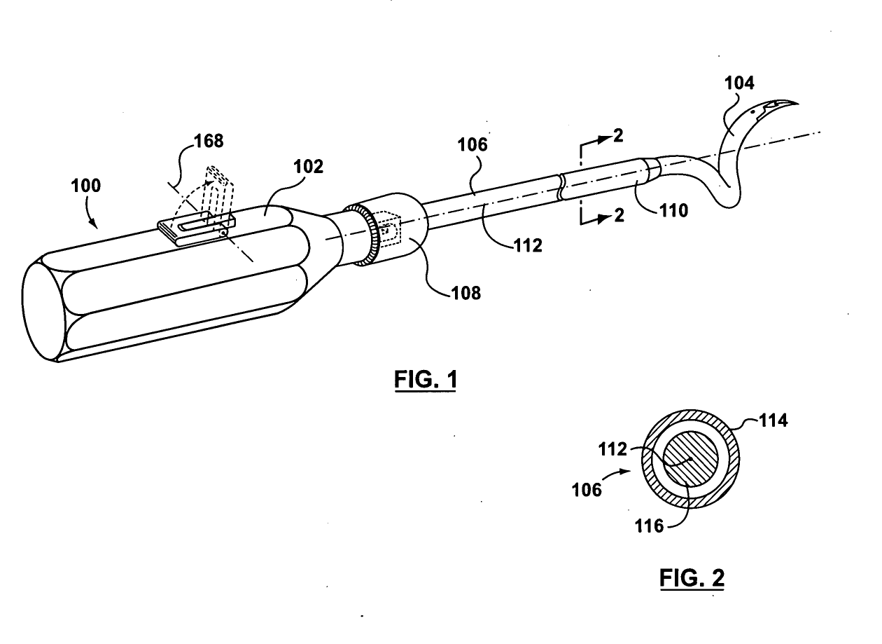

Referring to FIG. 1, a suturing instrument for minimally invasive surgery (MIS) is shown generally at reference character 100. The instrument 100 comprises a handle 102, a tip 104, and a shank 106 extending between the handle 102 and the tip 104.

The shank 106 has a handle end 108 connectable to the handle 102 and a tip end 110 joined to the tip 104. In the example illustrated, the shank 106 is releasably attached to the handle 102 at the handle end 108, and the tip 104 is integrally formed with the shank 106. The shank 106 and tip 104 together form a head of the instrument 100. Releasable attachment of the head to the handle 102 can allow for interchangeability of any one of a variety of heads with handles 102.

In the example illustrated, the shank 106 is generally straight along its length, extending lengthwise along a longitudinal axis 112 between the handle end 108 and the tip end 110. The shank 106 is, in the example illustrated, round in cross-section (FIG. 2), and has a generally cylindrical outer surface 114. The outer surface 114 can taper inwardly along all or a portion of its length in a direction from the handle end 108 to the tip end 110. The shank 106 is, in the example illustrated, hollow and houses an actuator 116 in its interior. Further details of the actuator 116 are described subsequently herein.

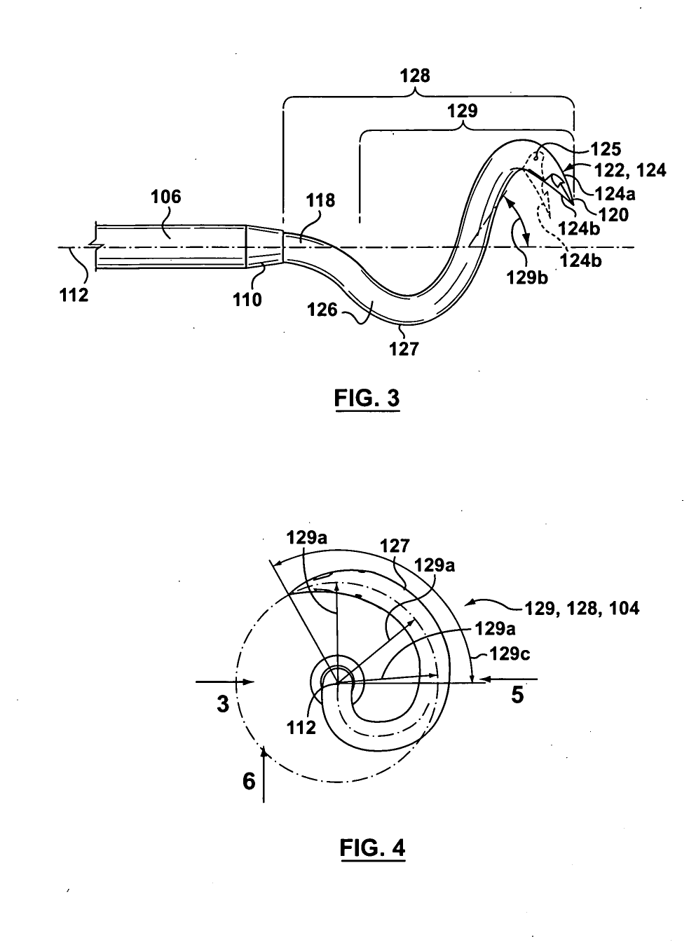

Referring to FIG. 3, the tip 104 comprises a proximal end 118 adjacent the tip end 110 of the shank 106, and a distal end 120 opposite the proximal end 118. The distal end 120 is pointed to facilitate penetrating tissue during use of the instrument 100. The tip 104 further comprises a gripper member 122 adjacent the distal end 120. In the example illustrated, the gripper member 122 comprises a pair of jaws 124, including a first (radially outer) jaw 124a and a second (radially inward) jaw 124b. The first jaw 124a is, in the example illustrated fixed relative to the tip 104. The second jaw 124b is, in the example illustrated, pivotable about a pivot axis 125 for moving the jaws 124 between open and closed positions. The pivot axis 125 in the example illustrated is shown schematically in the drawings (for clarity) as including a pin member passing through the jaws and with ends visible externally of the tip 104. Other joint constructions for providing the pivot axis 125 are also possible, such as, for example, joint constructions in which a pin (or similar member) is housed internally of the jaws without intersecting or interrupting an outer surface thereof.

The tip 104 comprises a slender body 126 that is, in the example illustrated, generally tubular. The tip body 126 has an outer tip surface 127 and a hollow interior for passage of the actuator 116 therethrough. The outer tip surface 127 is, in the example illustrated, generally round in cross-section.

The tip 104 comprises a compound curve 128 along its length between the proximal and distal ends 118, 120. The compound curve 128 can comprise at least three curve elements, such that at any orientation of the tip 104, the projection of the tip onto each of three mutually orthogonal planes is curved in shape. The compound curve 128 is a curve in three-dimensional space.

In the example illustrated, the compound curve 128 of the tip 104 includes a generally helical portion 129 extending from the distal end 120 to a point near or at the proximal end 118 of the tip 104. The helical portion 129 of the compound curve 128 is, in the example illustrated, coaxial with the longitudinal axis 112 of the instrument 100. The helical portion 129 has a generally constant curvature, with a helix radius 129a defined by a radial distance between the tip 104 and the longitudinal axis 112 that is generally constant along the length of the helical portion 129 (FIG. 4). The helical portion 129 has a circumferential extent 129c that is at least about 90 degrees (about the axis 112), and can be 180 degrees or more. In the example illustrated, the circumferential extent 129c is about 135 degrees. In the example illustrated, the helical portion 129 of the compound curve 128 follows a right-handed helix shape, with a helix angle 129b of about 45 degrees (FIG. 3).

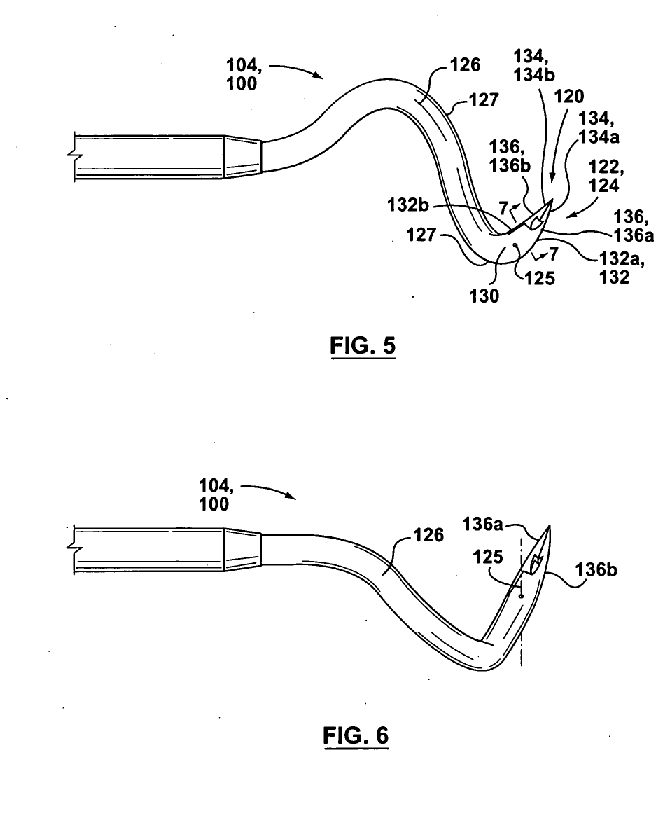

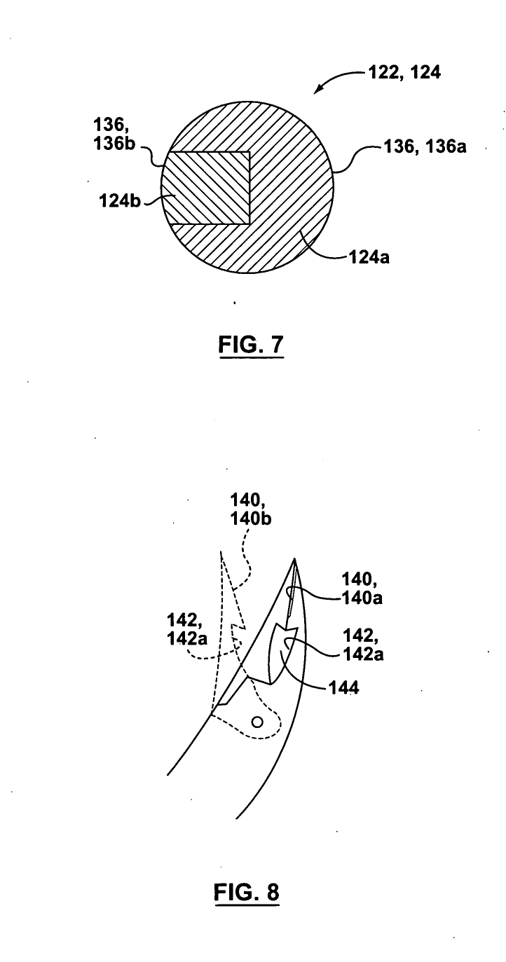

Referring to FIGS. 5 and 6, the gripper member 122 is, in the example illustrated, shaped to blend with the outer tip surface 127 of the body 126 of the tip 104. Each of the jaws 124 (i.e. jaw 124a and 124b) has a root end 132 (132a and 132b, respectively) where the jaw 124 is connected to a jaw-adjoining portion 130 of the tubular body 126 of the tip 104, and a pointed end 134 (134a, 134b, respectively) at the distal end 120 of the tip 104. Each jaw 124 further has an outer jaw surface 136 that is generally semi-circular in cross-section (FIG. 7), having a radius at the root end 132 generally equal to the radius of the jaw adjoining portion 130 of the tubular body 126 of the tip 104.

In the lengthwise direction (generally along the length of the tip 104), the outer jaw surface 136 can be curved to blend with, and/or form an extension of, the compound curve 128. In the example illustrated, and as viewed in a plane generally perpendicular to the pivot axis 125 (FIG. 5), the outer jaw surface 136a of the first jaw 124a has a convex profile from the root 132a to the pointed end 134a. The outer jaw surface 136b of the second jaw 124b has a concave profile from the root 132b to the pointed end 134b. The outer surfaces 136a, 136b generally follow the same helical path defined by the helical portion 129 of the compound curve 128 (although the surfaces do converge at the point 120). In the example illustrated, the outer surfaces 136a, 136b can also have lengthwise convex and concave portions when viewed in a plane generally parallel to both the pivot axis 125 and the longitudinal axis 112 (FIG. 6).

Referring to FIG. 8, the jaws 124 have inner jaw surfaces 140 generally opposite the outer jaw surfaces 136. The inner jaw surfaces 140 of at least one of the jaws 124 can be notched (e.g. at notch 142) to provide a retaining aperture 144 between the jaws 124 when the jaws 124 are in the closed position. In the example, illustrated, each of the jaws 124a, 124b has a notch 142a, 142b, the notches 142 cooperating to form the retaining aperture 144 when the jaws 124 are in the closed position. The retaining aperture 144 can accommodate the thickness of a suture without impeding full closure of the jaws 124.

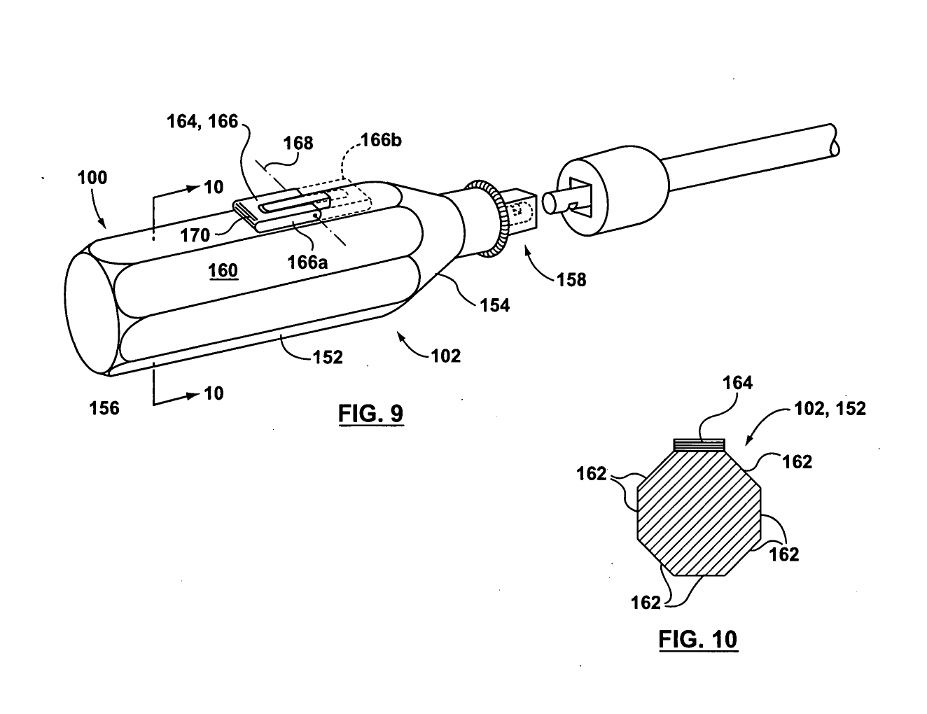

Referring now also to FIG. 9, the handle 102 in the illustrated example has a handle body 152 that is sized to fit within the hand of a user. The body 152 has a front end 154 and a back end 156 opposite the front end 154. The front end 154 can be provided with a coupling 158 for releasable attachment to the shank 106 of the head of an instrument 100. The handle body 152 can include a gripping surface 160 extending generally along at least a portion of the body 152 between the front and back ends 154, 156. The gripping surface 160 can facilitate manipulating the handle body 152 when in use, including for example, rotating the handle body 152 generally about the longitudinal axis 112. In the example illustrated, the gripping surface 160 comprises lands 162 (or flat surfaces) arranged to give the handle body 152 a polygonal shape in cross section. In the example illustrated, the handle body 152 has an octagonal shape (FIG. 10).

The handle 102 further includes a driver element 164 coupled to the actuator 116 for moving the jaws 124 between the open and closed positions. In the example illustrated, the driver element 164 comprises a lever 166 that can be moved between advanced and retracted positions 166a, 166b, for example, by pivoting the lever 166 about a lever axis 168. In the example illustrated, the lever axis 168 is oriented generally orthogonal to the longitudinal axis 112. The lever 166 can comprise an engagement edge 170 that is positioned proud of the outer gripping surface 160 of the handle, and that can be contoured and/or textured to facilitate engagement by, for example, the thumb of a user of the instrument 100.

In the example illustrated, the lever 166 is self-locking (for example by releasable engagement of a detent element-not shown) when in the advanced position 166a and when in the retracted position 166b. The lever 166 has a relatively thin profile so that it presents minimal interference to a user's hand (relative to the surrounding handle portions) when, for example, the handle 102 is rotated about the longitudinal axis 112 of the instrument 100 during use. In the example illustrated, the lever 166 is generally flush against one of the lands 162 of the handle 102 when in the advanced and retracted positions 166a, 166b.

The driver element 164 is coupled to the actuator 116 so that movement of the driver element 164 causes, in the example illustrated, lengthwise movement of the actuator relative to the shank 106 and tip 104. The actuator 116 can include an elongate element that is generally inextensible along its length for positive motion transfer to the jaws, and sufficiently lateral flexible or bendable to move through the compound curve of the tip. In the example illustrated, the actuator 116 comprises a flexible steel band that is slidable along the length of the tip 104. In other examples, the actuator 116 can comprise a length of braided strands of material such as steel or synthetic fibers. The outer surface of the actuator 116, and/or the inner surface of the shank and tip can be coated or lined with a friction-reducing material (e.g. graphite) to reduce friction when the actuator 116 moves relative to the shank 106 and tip 104.

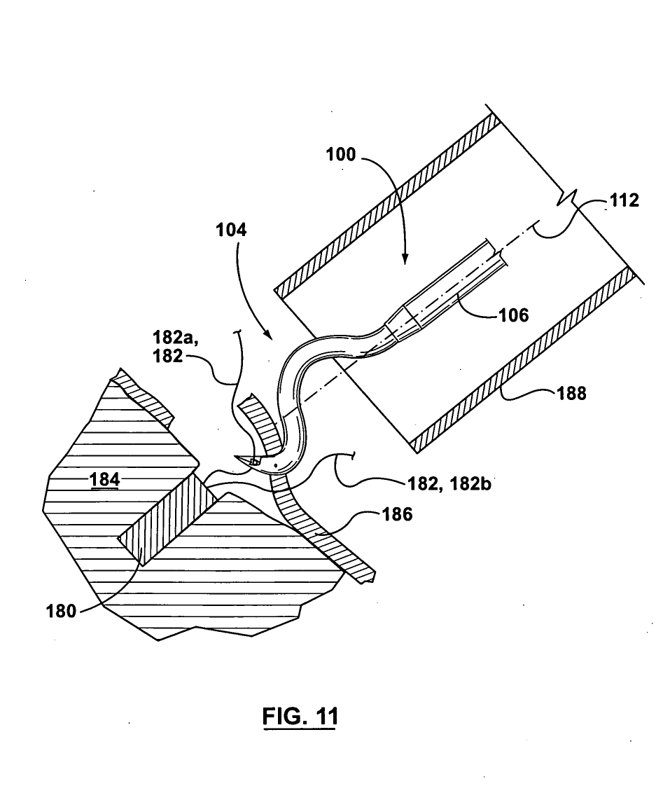

Referring to FIG. 11, an example of the use of the instrument 100 will be described in relation to a tissue-to-bone repair procedure. In the example procedure, a suture anchor 180 with two tails 182 has been installed in bone 184 adjacent the tissue 186 to be repaired. Based on the position of the anchor 180, the tissue 186, and the cannula 188, the instrument 100 with a particular tip 104 and compound curve 128 is selected. In other situations, an alternate head having a tip with a different curve configuration (e.g. different helix angle, opposite hand helix) may be selected.

The instrument 100 is introduced into the patient's body through the cannula 188, with the tip 104 facing the tissue 186 opposite a first one of the suture tails 182a. With the jaws 124 in the closed position, the sharp point is, in the example illustrated, inserted into the tissue 186 at a desired exit point for the suture 182a. The tip 104 of the instrument 100 is urged through the tissue 186, towards the first suture tail 182a, generally by rotating the instrument 100 in a clockwise direction about the axis 112.

Once the tip 104 has passed through the tissue 186 far enough so that the pivot axis 125 has passed completely through the tissue 186, the jaws 124 are moved to the open position and are passed over the first suture tail 182a. The opened jaws 124 are passed on either side of the suture 182a until the notches 142 are aligned with the first suture 182a. The jaws can then be closed, with the first suture 182a trapped in the retaining aperture 144 formed by the notches 142. The tip 104 can then be withdrawn by counter-clockwise rotation about the axis 112, pulling the suture 182a through the channel formed during the previous penetration of the tissue 186 by the tip 104. The first suture 182a can then be tied to the second suture 182b, pulling the tissue 186 snugly against the bone 184.

In other examples, the suture tail 182a can be gripped in the jaws prior to penetrating the tissue, so that the suture tail is carried through the tissue when the instrument penetrates the tissue, rather than when the instrument is withdrawn from the tissue. In either case, the movement of the instrument 100 through the tissue 186 can primarily be effected by rotation of the instrument 100 about the shank axis 112, with the helical portion 129 of the instrument 100 advancing and withdrawing through the tissue 186. The shank axis 112 can advantageously be oriented generally collinear with the axis of the cannula 188, and the limited space available in the MIS procedure can be used with a high degree of efficiency.

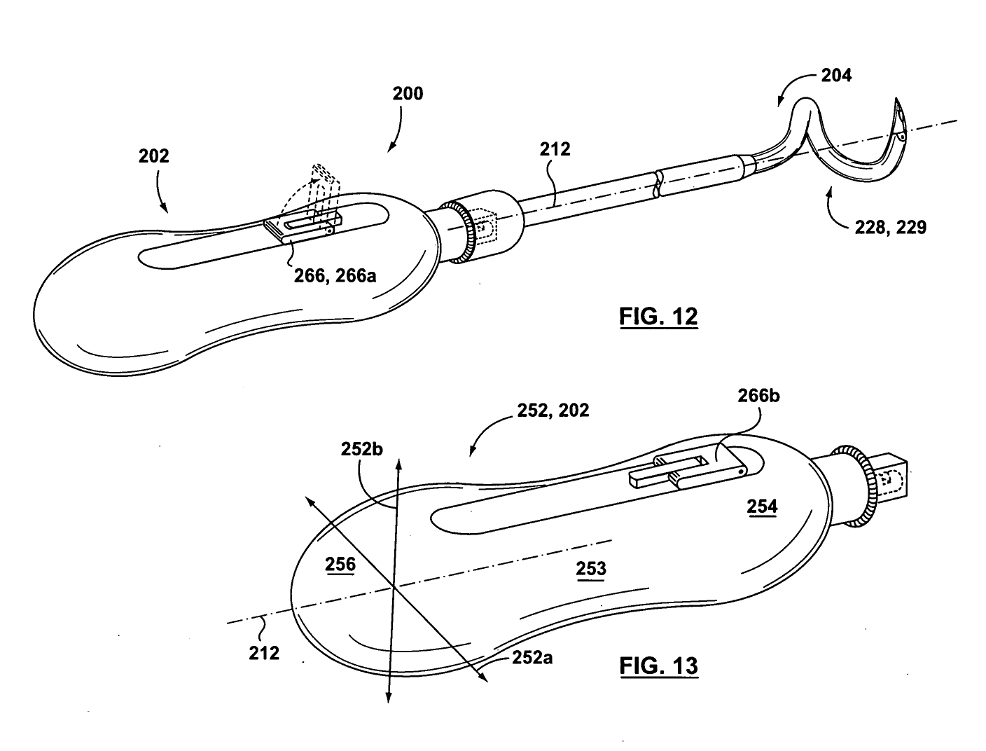

Referring now to FIGS. 12 and 13, another example of a suturing instrument 200 is described. The suturing instrument 200 is similar to the instrument 100, with similar features identified by like reference characters, incremented by 100.

The suturing instrument 200 comprises a handle 202 that has a body 252 that is generally peanut shaped, having a narrow intermediate portion 253 disposed between enlarged front and back ends 254, 256. The outer gripping surface 260 is not symmetrical about the axis 212 of the instrument, but instead is flattened, with the body 252 having a major axis 252a and a minor axis 252b, each normal to each other and the axis 212. The increased width of the handle body 252 along the major axis 252a can provide increased leverage when rotating the instrument 200 about the axis 212 during use, i.e. particularly when penetrating tissue.

The driver element 264 of the handle 202 is coupled to the actuator 216 for moving the jaws 224 between the open and closed positions. In the example illustrated, the driver element 264 comprises a lever 266 that can be moved between advanced and retracted positions 266a, 266b, and that is self-locking (for example by releasable engagement of a detent element-not shown) when in the advanced position 266a (FIG. 12) and when in the retracted position 266b (FIG. 13).

The tip member 204 has a slender body that is curved along at least a portion of its length in the form of a compound curve 228. At least a portion of the compound curve 228 is helical, providing the tip 204 with a helical portion 229. The helical portion 229 is, in the example illustrated, a left-handed helix, having a helix axis coincident with the longitudinal axis 212, a helix radius 229a of about 8 to 12 mm, and a helix angle 229b of about 20 degrees.

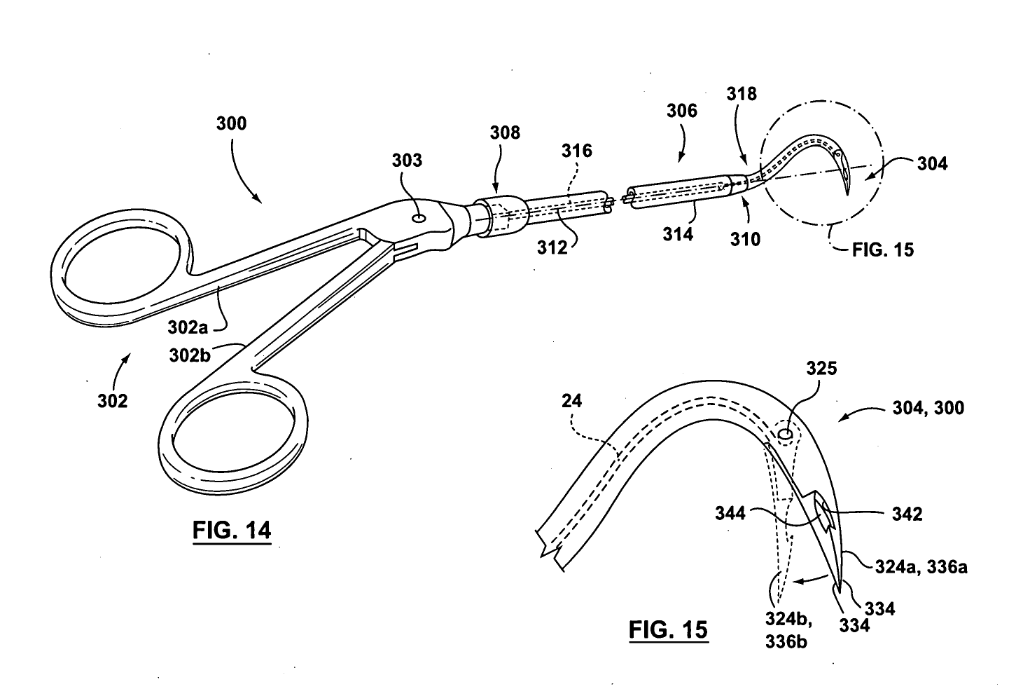

Referring now to FIG. 14, another example of a suturing instrument 300 is described. The suturing instrument 300 is similar to the instrument 100, with similar features identified by like reference characters, incremented by 200.

The suturing instrument 300 includes a familiar scissors-style handle 302 with arms 302a and 302b that are pivotable relative to each other about a handle pivot 303. Pivoting the arms 302a, 302b apart can urge the actuator 316 forward, opening the jaws 324. Pivoting the handles together can draw the actuator 316 back (towards the handle 302) to close the jaws 324.

The compound curve 328 of the instrument 300 comprises, in the example illustrated, a first curve element 328a starting at the proximal end 318 and directing the tubular body downward (in FIG. 17) from the shank axis 312. The compound curve 328 further comprises a second curve element 328b extending from the first curve element 128a and directing the tubular body 326 back up (in FIG. 17) towards (and across) the shank axis 312. The compound curve 328 further comprises a third curve element 328c directing the tubular body rightwards of the shank axis 312 (when viewed from above, along the shank axis 312 from the handle end towards the tip end 310).

In the longitudinal direction, the outer jaw surface 336 can be curved to blend with one or more curve elements 328a, 328b, 328c of the compound curve 328. In the example illustrated, the outer jaw surface 336a of the first (or outermost) jaw 336a has a convex profile from the root 332 to the pointed end 334. The outer jaw surface 336b of the second (or innermost) jaw 324b has a concave profile from the root 332 to the pointed end 334. The concave and convex profiles of the outer surfaces 336a, 336b extend in a plane generally perpendicular to the axis 325 about which the jaws 324 pivot.

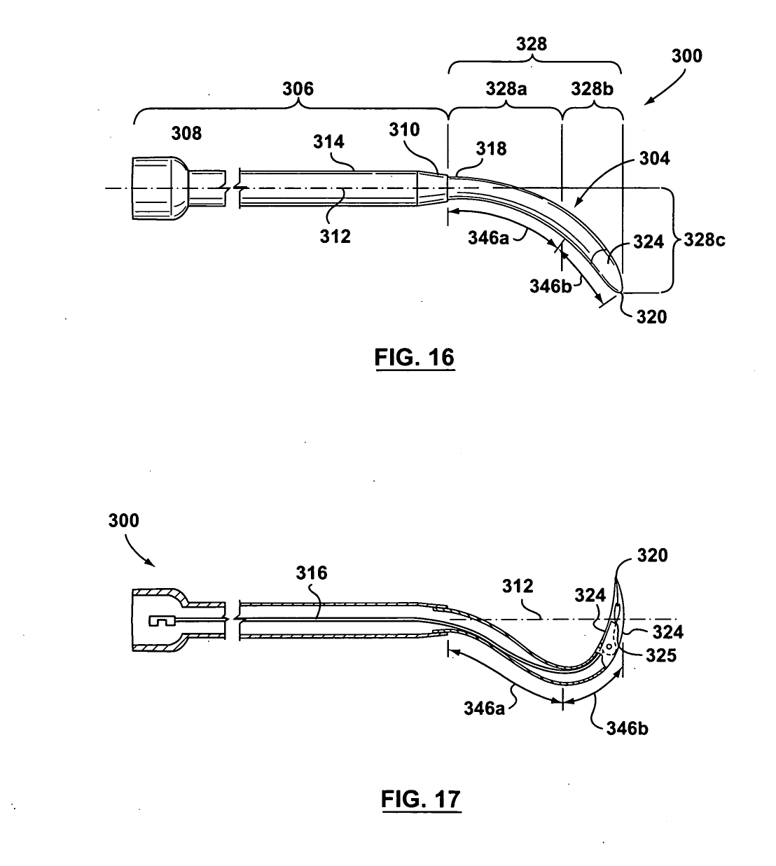

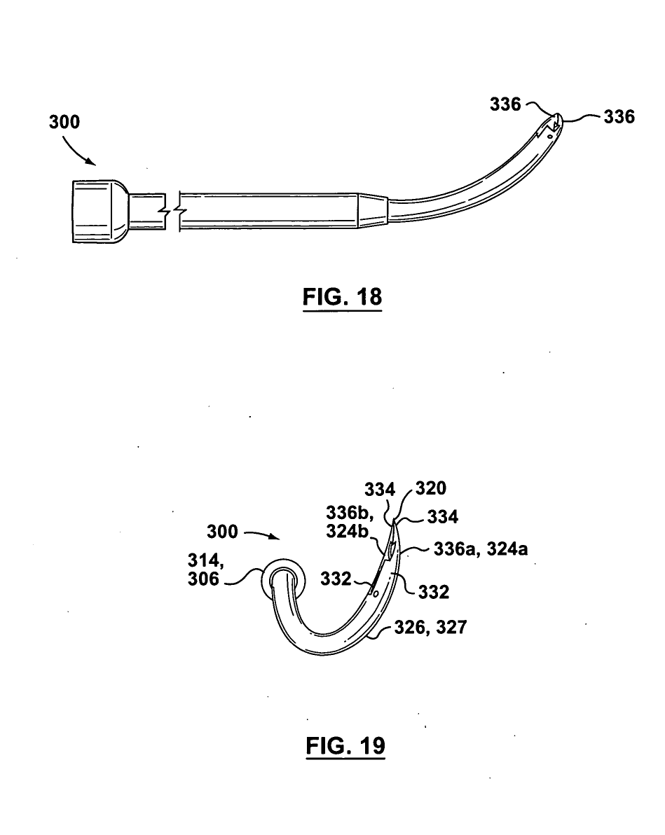

Referring again to FIGS. 16 and 17, the tip 304 in the example illustrated comprises two tip segments 346a, 346b along its length. The first tip segment 346a extends away from the tip end 308 of the shank 306 along a first segment path 348a directed obliquely away from the shank axis 312. In the example illustrated, the first segment path 348a diverges away from the longitudinal axis 312 when viewed as a projection in both a first (vertical) plane, and a second (horizontal) plane orthogonal to the first plane (this can be seen in the projection of the tip 304 in the vertical plane of FIG. 17 and in the horizontal plane of FIG. 16).

The second tip segment 346b extends further away from the tip end 308 of the shank 306 along a second segment path 348b that is directed (at least when viewed as a projection in one plane) obliquely towards the shank axis 312. In the example illustrated, the second tip segment 346b converges towards (and in fact crosses over) the shank axis 112 when viewed as a projection in a vertical plane (FIG. 17), and diverges further away from the shank axis 312 when viewed as a projection in a horizontal plane (FIG. 16). Accordingly, in the example illustrated, the first curve element 328a of the compound curve 328 comprises the first tip segment 346a, the second curve element 328b comprises the second tip segment 346b, and the third curve element 328c comprises the first and second tip segments 346a, 346b.

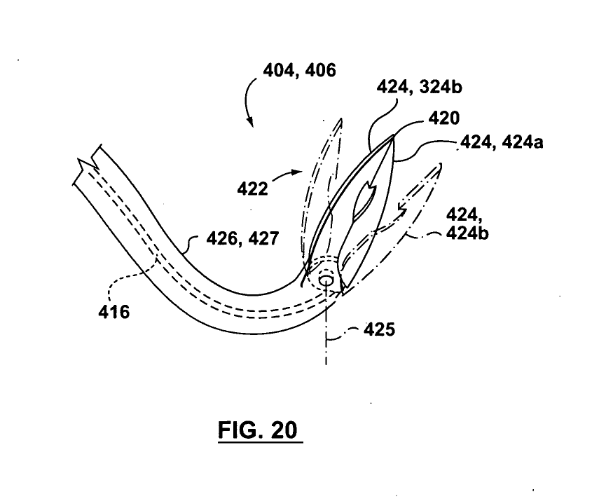

Referring now to FIG. 20, another example of suturing instrument 400 having a tip 404 is illustrated. The instrument 404 is similar to the instrument 100, and like features are identified by like reference characters, incremented by 300.

The tip 404 comprises a gripper member 422 including a pair of jaws 424 (424a, and 424b). In the instrument 400, both jaws 424a and 424b are movable about a jaw pivot axis 425.

While the above description provides examples of one or more processes or apparatuses, it will be appreciated that other processes or apparatuses may be within the scope of the accompanying claims.

I claim:

1. An instrument for minimally invasive surgery, comprising:
a) a shank attachable to a handle, the shank extending lengthwise along a shank axis between a handle end and a tip end;
b) a tip member joined to the tip end of the shank, the tip member extending along a tip length between a proximal end adjacent the tip end of the shank and a distal end opposite the proximal end, the tip member comprising a jaw adjacent the distal end, the jaw movable between open and closed positions, the tip member comprising an outer surface that converges, at least when the jaw member is in the closed position, to a sharp point at the distal end to facilitate penetration through tissue, the tip member being formed in the shape of a compound curve along at least a portion of the tip length; and
c) an actuator coupled to the jaw member and extending lengthwise of the instrument to the handle end of the shank.

2. The instrument of claim 1, wherein the compound curve comprises at least a portion of a helix defining a helical portion of the tip.

3. The instrument of claim 2, wherein the helical portion has a helix axis generally collinear with the shank axis.

4. The instrument of claim 2, wherein the helical portion has a helix angle within a range from about 15 degrees to about 90 degrees.

5. The instrument of claim 2, wherein the helical portion has a helix radius within a range from about 4 mm to about 10 mm.

6. The instrument of claim 2, wherein the helical portion extends at least about 180 degrees about the shank axis.

7. The instrument of claim 2, wherein the helical portion comprises the jaw member.

8. The instrument of claim 7, wherein the helical portion comprises the pointed distal end of the tip member.

9. The instrument of claim 8, wherein the jaw member comprises an outer jaw surface that is tapered at one end, the pointed distal end of the tip member comprising said tapered one end.

10. The instrument of claim 1, wherein the tip member comprises a generally tubular body extending between the proximal end and the jaw member.

11. The instrument of claim 10, wherein the actuator is housed within the tubular body.

12. The instrument of claim 11, wherein the actuator is movable with respect to the tip for moving the jaws between the open and closed positions.

13. The instrument of claim 11, wherein the actuator is slidable relative to the tip.

14. The instrument of claim 10, wherein actuator comprises an elongate element generally inextensible along its length and laterally flexible.

15. An instrument for minimally invasive surgery, comprising:
a) a shank extending lengthwise along a longitudinal axis between a handle end and a tip end;
b) a tip joined to the tip end of the shank, the tip comprising a slender body extending between a proximal end adjacent the tip end of the shank and a distal end opposite the proximal end, at least a portion of the body curved along a helical path defining a helical portion of the tip, the tip comprising a jaw member adjacent the distal end, the jaw member movable between open and closed positions, the tip comprising an outer surface that converges, at least when the jaw member is in the closed position, to a sharp point opposite the tip end of the shank to facilitate penetration through tissue; and
c) an actuator coupled to the jaw member and extending lengthwise of the instrument to the handle end of the shank.

16. The instrument of claim 15, wherein the helical portion of the tip comprises the jaw member.

17. The instrument of claim 16, further comprising a handle attachable to the handle end of the shank.

18. The instrument of claim 17, wherein the handle comprises a driver element mounted to the handle and coupled to the actuator, the driver element movable between advanced and retracted positions corresponding to the closed and open positions of the jaws.

19. A method of passing a suture through tissue, comprising:
a) providing an instrument having a shank extending along a longitudinal axis and a tip member joined to the shank, the tip member including a jaw at a distal end of the tip member opposite the shank, the jaw movable between open and closed positions, the tip member having an outer surface that converges to a point at least when the jaw is in the closed position, the tip member having a length at least a portion of which is curved in a helical path about the longitudinal axis;
b) with the jaw in the closed position, rotating the instrument in a first rotary direction about the longitudinal axis to urge the tip through the tissue;
c) when the jaw has passed through the tissue, opening the jaw and either releasing a passed suture or approaching an unpassed suture;
d) closing the jaw; and
e) rotating the instrument in a second rotary direction opposite the first rotary direction to withdraw the tip through the tissue.

20. Use of the instrument of claim 1 to pass a suture through tissue.
